(19) United States
(12) Patent Application Publication
(10) Pub. No.: US 2021/0231624 A1
(43) Pub. Date: Jul. 29, 2021

(54) AZOCALIXARENE PROBE AND ITS USE FOR DETECTING CARBON DIOXIDE

(71) Applicant: The University of Surrey, Surrey (GB)

(72) Inventors: Angela F. DANIL DE NAMOR, Surrey (GB); Nawal AL HAKAWATI, Surrey (GB)

(21) Appl. No.: 17/050,915

(22) PCT Filed: May 2, 2019
(86) PCT No.: PCT/GB2019/051225

§ 371 Date: Oct. 27, 2020

(30) Foreign Application Priority Data
May 2, 2018 (GB) 1807212.4

(51) Int. Cl. G01N 31/22; C07C 245/08

(52) U.S. Cl. CPC G01N 31/224; C07C 245/08

(57) ABSTRACT

The disclosure describes a probe comprising an azo-calixarene complexed with an anion. The anion may be a fluoride ion or a carbonate ion. The probe may be used to sense and/or capture carbon dioxide.

### CROSS-REFERENCE

This application is a 371 National Stage filing and claims the benefit under 35 U.S.C. § 120 to International Application No. PCT/GB2019/051225, filed May 2, 2019, which claims priority to Great Britain Application No. 1807212.4, filed May 2, 2018, each of which is incorporated herein by reference in its entirety.

The present invention relates to a probe, and more specifically a probe configured to sense and/or capture carbon dioxide. The disclosure also extends to a method of producing the probe and uses of the probe.

The ability to sense carbon dioxide (CO₂) is important in a number of applications. For instance, it allows the quality of air in an environment to be measured. To enable a person to quickly and accurately determine whether or not CO₂ is present, a sensor should ideally provide a signal which is visible to the naked eye when CO₂ is detected.

Furthermore, since CO₂ is a greenhouse gas, the ability to capture CO₂ and thereby prevent it from being released to the atmosphere is also desirable. Carbon capture technology is particularly desirable for use in power plants or mining applications. It will be appreciated that over time a device configured to capture CO₂ will become saturated. Accordingly, to reduce costs, it is desirable that the device allows recovery of the CO₂ gas so that the device can be reused.

Many existing devices only allow the recovery of CO₂ when they are heated to elevated temperatures, requiring a large amount of energy to be used and reducing the overall efficiency of the process.

The present invention arises from the inventors work in attempting to overcome the problems associated with the prior art.

In accordance with a first aspect, there is provided a probe comprising an azo-calixarene complexed with an anion.

Advantageously, the probe changes colour when exposed to carbon dioxide, providing a visible indication that it is present. Furthermore, the captured CO₂ can be recovered from the probe without the need to use elevated temperatures enabling the probe to be used multiple times.

Preferably, the probe comprises at least 1 mole of anion for each mole of azo-calixarene, more preferably at least 5, 10, 15, 20 or 25 moles of anion for each mole of azo-calixarene and most preferably at least 50 or 75 moles of anion for each mole of azo-calixarene.

The anion may be a fluoride ion or a carbonate ion.

In some embodiments, the anion is a fluoride ion. Preferably, the probe comprises at least 1 mole of fluoride for each mole of azo-calixarene, more preferably at least 5, 10, 15, 20 or 25 moles of fluoride for each mole of azo-calixarene and most preferably at least 50 or 75 moles of fluoride for each mole of azo-calixarene.

The probe may comprise a counter ion. The counter ion may comprise an ammonium ion.

The ammonium ion may be expressed as (R¹³)₄N⁺ where each R¹³ group is independently a C₁-C₁₂ alkyl. In one embodiment, each R¹³ group is independently a C₁-C₆ alkyl. Accordingly, each R¹³ group may independently be methyl, ethyl, propyl, butyl, pentyl or hexyl. In a preferred embodiment, each R¹³ group is n-butyl. Accordingly, the ammonium ion may have formula (C₄H₉)₄N⁺.

Alternatively or additionally, the counter ion may comprise a cation of an alkali metal or an alkaline earth metal. The cation of the alkali metal may be a lithium ion, a sodium ion or a potassium ion. Preferably, the cation of the alkali metal is a sodium ion or a potassium ion.

The azo-calixarene complexed with the anion may be dissolved in a solvent. The azo-calixarene complexed with the fluoride ion may be dissolved in a solvent. The solvent may comprise dimethyl sulfoxide (DMSO), N,N-dimethylformamide (DMF), tetrahydrofuran (THF) or chloroform (CHCl₃).

The amount of the azo-calixarene dissolved in the solvent may comprise a concentration of at least 1×10⁻¹⁰ mol dm⁻³ of the azo-calixarene, more preferably at least 1×10⁻⁹ mol dm⁻³, at least 1×10⁻⁸ mol dm⁻³ or at least 1×10⁻⁷ mol dm⁻³ of the azo-calixarene, and most preferably at least 1×10⁻⁶ mol dm⁻³ of the azo-calixarene. The amount of the azo-calixarene dissolved in the solvent may comprise a concentration of less than 0.1 mol dm⁻³ of the azo-calixarene, more preferably less than 5×10⁻² mol dm⁻³, less than 1×10⁻¹ mol dm⁻³ or less than 5×10⁻³ mol dm⁻³ of the azo-calixarene, and most preferably less than 1×10⁻³ mol dm⁻³ of the azo-calixarene. The amount of the azo-calixarene dissolved in the solvent may comprise a concentration of between 1×10⁻¹⁰ and 0.1 mol dm⁻³ of the azo-calixarene, more preferably between 1×10⁻⁹ and 5×10⁻² mol dm⁻³, between 1×10⁻⁸ and 1×10⁻² mol dm⁻³ or between 1×10⁻⁷ and 5×10⁻³ mol dm⁻³, and most preferably between 1×10⁻⁶ and 1×10⁻³ mol dm⁻³ of the azo-calixarene.

The amount of the anion dissolved in the solvent may comprise a concentration of at least 1×10⁻⁸ mol dm⁻³ of the anion, more preferably at least 1×10⁻⁷ mol dm⁻³, at least 1×10⁻⁶ mol dm⁻³ or at least 1×10⁻⁵ mol dm⁻⁴ of the anion, and most preferably at least 1×10⁻³ mol dm⁻³ of the anion. The amount of the anion dissolved in the solvent may comprise a concentration of less than 1 mol dm⁻³ of the anion, more preferably less than 0.75 mol dm⁻³, less than 0.5 mol dm⁻³ or less than 0.25 mol dm⁻³ of the anion, and most preferably less than 0.1 mol dm⁻³ of the anion. The amount of the anion dissolved in the solvent may comprise a concentration of between 1×10⁻⁸ and 1 mol dm⁻³ of the anion, more preferably between 1×10⁻⁷ and 0.75 mol dm⁻³, between 1×10⁻⁶ and 0.5 mol dm⁻³ or between 1×10⁻⁵ and 0.25 mol dm⁻⁴ of the anion, and most preferably between 1×10⁻³ and 0.1 mol dm⁻³ of the anion.

The amount of the fluoride dissolved in the solvent may comprise a concentration of at least 1×10⁻⁸ mol dm⁻³ of the fluoride, more preferably at least 1×10⁻⁷ mol dm⁻³, at least 1×10⁻⁶ mol dm⁻³ or at least 1×10⁻⁵ mol dm⁻⁴ of the fluoride, and most preferably at least 1×10⁻³ mol dm⁻³ of the fluoride. The amount of the fluoride dissolved in the solvent may comprise a concentration of less than 1 mol dm⁻³ of the fluoride, more preferably less than 0.75 mol dm⁻³, less than 0.5 mol dm⁻³ or less than 0.25 mol dm⁻³ of the fluoride, and most preferably less than 0.1 mol dm⁻³ of the fluoride. The amount of the fluoride dissolved in the solvent may comprise a concentration of between 1×10⁻⁸ and 1 mol dm⁻³ of the fluoride, more preferably between 1×10⁻⁷ and 0.75 mol dm⁻³, between 1×10⁻⁶ and 0.5 mol dm⁻³ or between 1×10⁻⁵ and 0.25 mol dm⁻⁴ of the fluoride, and most preferably between 1×10⁻³ and 0.1 mol dm⁻³ of the fluoride.

It may be appreciated that in embodiments where the counter ion has a single positive charge and the anion has a single negative charge, or where the counter ion has a double positive charge and the anion has a double negative charge, then the counter ion may be present at the same concentration as the anion. Alternatively, in embodiments where the counter ion has a double positive charge and the anion has a single negative charge, then the counter ion may be present at half the concentration as the fluoride. Similarly, in embodiments where the counter ion has a single positive charge and the anion has a double negative charge, then the counter ion may be present at twice the concentration as the fluoride.

For instance, in embodiments where the anion is fluoride and the counter ion has a single positive charge, then the counter ion may be present at the same concentration as the fluoride. Alternatively, in embodiments where the anion is fluoride and the counter ion has a double positive charge, then the counter ion may be present at half the concentration as the fluoride.

Alternatively, the probe may comprise a solid. The solid may be provided in the form of a sheet. Alternatively, the solid may be provided in the form of one or more pellets.

In one embodiment, the azo-calixarene is an azo-calix[4]arene.

Preferably, the azo-calixarene is a compound of formula (I):

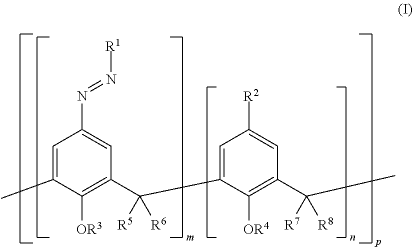

wherein R¹ is selected from the group consisting of hydrogen, an optionally substituted C₁-C₁₀ alkyl, an optionally substituted C₂-C₁₀ alkenyl, an optionally substituted C₂-C₁₀ alkynyl, NR¹¹R¹², an optionally substituted C₅-C₁₀ aryl and an optionally substituted 3 to 10 membered heteroaryl;

R² is selected from the group consisting of hydrogen, an optionally substituted C₁-C₁₀ alkyl, an optionally substituted C₂-C₁₀ alkenyl, an optionally substituted C₂-C₁₀ alkynyl, a halogen, OH, (CR⁹R¹⁰)ₐSO₂OR⁹, (CR⁹R¹⁰)ₐSO₂NR⁹R¹⁰, NO₂, (CR⁹R¹⁰)ₐPO₂OH, (CR⁹R¹⁰)ₐCOOR⁹, (CR⁹R¹⁰)ₐSS(CR⁹R¹⁰)_{b}COOR⁹, (CR⁹R¹⁰)ₐNR⁹R¹⁰ and N═NR⁹; R³ and R⁴ are each independently selected from the group consisting of hydrogen, an optionally substituted C₁-C₁₀ alkyl, an optionally substituted C₂-C₁₀ alkenyl, an optionally substituted C₂-C₁₀ alkynyl, (CR⁹R¹⁰)ₐNR⁹R¹⁰, (CR⁹R¹⁰)ₐOR⁹, (CR⁹R¹⁰)ₐCOR⁹, (CR⁹R¹⁰)ₐCOOR⁹, (CR⁹R¹⁰)ₐCONR⁹R¹⁰, (CR⁹R¹⁰)ₐSO₂OR⁹, (CR⁹R¹⁰)ₐCOO(CR⁹R¹⁰)_{b}OR⁹, (CR⁹R¹⁰)ₐCOO(CR⁹R¹⁰)_{b}COR⁹ and (CR⁹R¹⁰)ₐCOO(CR⁹R¹⁰)_{b}SR⁹;

R⁵, R⁶, R⁷ and R⁸ are each independently selected from the group consisting of hydrogen, a halogen, an optionally substituted C₁-C₁₀ alkyl, an optionally substituted C₂-C₁₀ alkenyl and an optionally substituted C₂-C₁₀ alkynyl;

the or each R⁹ and R¹⁰ are independently selected from the group consisting of hydrogen, an optionally substituted C₁-C₁₀ alkyl, an optionally substituted C₂-C₁₀ alkenyl, an optionally substituted C₂-C₁₀ alkynyl, NR¹¹R¹², an optionally substituted C₅-C₁₀ aryl and an optionally substituted 3 to 10 membered heteroaryl;

an optionally substituted C₅-C₁₀ aryl or an optionally substituted 3 to 10 membered heteroaryl is unsubstituted or substituted with one or more substituents selected from the group consisting of an optionally substituted C₁-C₁₀ alkyl, an optionally substituted C₂-C₁₀ alkenyl, an optionally substituted C₂-C₁₀ alkynyl, a halogen, OR¹¹, NO₂, CN, COOR¹¹ and NR¹¹R¹²;

each R¹¹ and R¹² are independently selected from the group consisting of hydrogen, an optionally substituted C₁-C₁₀ alkyl, an optionally substituted C₂-C₁₀ alkenyl and an optionally substituted C₂-C₁₀ alkynyl;

an optionally substituted C₁-C₁₀ alkyl, an optionally substituted C₂-C₁₀ alkenyl or an optionally substituted C₂-C₁₀ alkynyl is unsubstituted or substituted with one or more substituents selected from the group consisting of halogen, OH, NH₂, CONH₂, COOH, CN, a C₅-C₁₀ aryl, a 3 to 10 membered heteroaryl, a C₃-C₆ cycloalkyl and a 3 to 8 membered heterocycle;

a and b are each independently an integer between 0 and 6; and

m is an integer between 1 and 8; n is an integer between 0 and 7; and p is an integer between 1 and 4; wherein the total of (m+n)xp is an integer between 4 and 8; or a salt, solvate or tautomeric form thereof

It will be appreciated that m may be 1, 2, 3, 4, 5, 6, 7 or 8, n may be 0, 1, 2, 3, 4, 5, 6 or 7 and p may be 1, 2, 3 or 4.

Accordingly, in on embodiment, m may be 1, n may be 1 and p may be 2, 3 or 4. In an alternative embodiment, m may be 1, n may be 3 and p may be 1 or 2.

In a preferred embodiment, m is an integer between 4 and 8, n is 0, and p is 1. Accordingly, in a preferred embodiment, the azo-calixarene is a compound of formula (II):

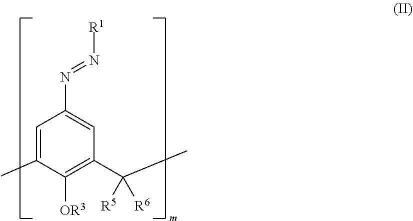

It will be appreciated that m may be 4, 5, 6, 7 or 8, more preferably 4, 6 or 8. Most preferably, m is 4.

Preferably, R¹ is an optionally substituted C₅-C₁₀ aryl or an optionally substituted 3 to 10 membered heteroaryl. In one embodiment, R¹ is an optionally substituted phenyl or an optionally substituted 6 membered heteroaryl. Accordingly, R¹ may be an optionally substituted phenyl or an optionally substituted pyridinyl. Most preferably, R¹ is an optionally substituted phenyl.

In one embodiment, R¹ is a C₅-C₁₀ aryl or a 3 to 10 membered heteroaryl wherein the aryl or heteroaryl are substituted with COOR¹¹ and/or NO₂. Preferably, R¹ is a C₅-C₁₀ aryl or a 3 to 10 membered heteroaryl wherein the aryl or heteroaryl are substituted with COOR¹¹ or NO₂. Preferably, the COOR¹¹ or NO₂ is in the para position.

Accordingly, R¹ may be

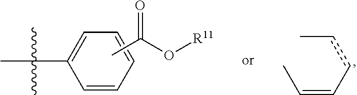

and more preferably is

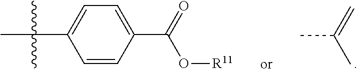

Preferably, R¹¹ is hydrogen, an optionally substituted C₁-C₁₀ alkyl, an optionally substituted C₂-C₁₀ alkenyl or an optionally substituted C₂-C₁₀ alkynyl. More preferably, R¹¹ is an optionally substituted C₁-C₅ alkyl, an optionally substituted C₂-C₅ alkenyl or an optionally substituted C₂-C₅ alkynyl. Most preferably, R¹¹ is an optionally substituted C₁-C₃ alkyl, an optionally substituted C₂-C₃ alkenyl or an optionally substituted C₂-C₃ alkynyl. Preferably, in embodiments where the alkyl, alkenyl or alkynyl is substituted, the substituent is selected from the group consisting of a halogen, OH and NH₂. The halogen may be fluorine, chlorine, bromine or iodine. Preferably, the halogen is fluorine.

R¹¹ may be methyl, ethyl, n-propyl, i-propyl, n-butyl or t-butyl. In a preferred embodiment, R¹¹ is ethyl.

In a most preferred embodiment, R¹ is

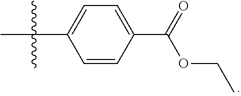 [SMILES: *c1ccc(C(=O)OCC)cc1]

Preferably, R³ is selected from the group consisting of hydrogen, an optionally substituted C₁-C₁₀ alkyl, an optionally substituted C₂-C₁₀ alkenyl and an optionally substituted C₂-C₁₀ alkynyl. More preferably, R³ is selected from the group consisting of hydrogen, an optionally substituted C₁-C₅ alkyl, an optionally substituted C₂-C₅ alkenyl and an optionally substituted C₂-C₅ alkynyl. Most preferably, R³ is selected from the group consisting of hydrogen, an optionally substituted C₁-C₃ alkyl, an optionally substituted C₂-C₃ alkenyl and an optionally substituted C₂-C₃ alkynyl. Preferably, in embodiments where the alkyl, alkenyl or alkynyl is substituted, the substituent is selected from the group consisting of a halogen, OH and NH₂. The halogen may be fluorine, chlorine, bromine or iodine. Preferably, the halogen is fluorine.

R³ may be methyl, ethyl, n-propyl, i-propyl, n-butyl or t-butyl. However, in a preferred embodiment, R³ is hydrogen.

Preferably, R⁵ and R⁶ are each independently selected from the group consisting of hydrogen, an optionally substituted C₁-C₁₀ alkyl, an optionally substituted C₂-C₁₀ alkenyl and an optionally substituted C₂-C₁₀ alkynyl. More preferably, R⁵ and R⁶ are each independently selected from the group consisting of hydrogen, an optionally substituted C₁-C₅ alkyl, an optionally substituted C₂-C₅ alkenyl and an optionally substituted C₂-C₅ alkynyl. Most preferably, R⁵ and R⁶ are each independently selected from the group consisting of hydrogen, an optionally substituted C₁-C₃ alkyl, an optionally substituted C₂-C₃ alkenyl and an optionally substituted C₂-C₃ alkynyl. Preferably, in embodiments where the alkyl, alkenyl or alkynyl is substituted, the substituent is selected from the group consisting of a halogen, OH and NH₂. The halogen may be fluorine, chlorine, bromine or iodine. Preferably, the halogen is fluorine.

R⁵ and R⁶ may independently be methyl, ethyl, n-propyl, i-propyl, n-butyl or t-butyl. In a preferred embodiment, R⁵ is methyl. In a preferred embodiment, R⁶ is methyl.

In a most preferred embodiment, the azo-calixarene is a compound of formula (III) or (VIII):

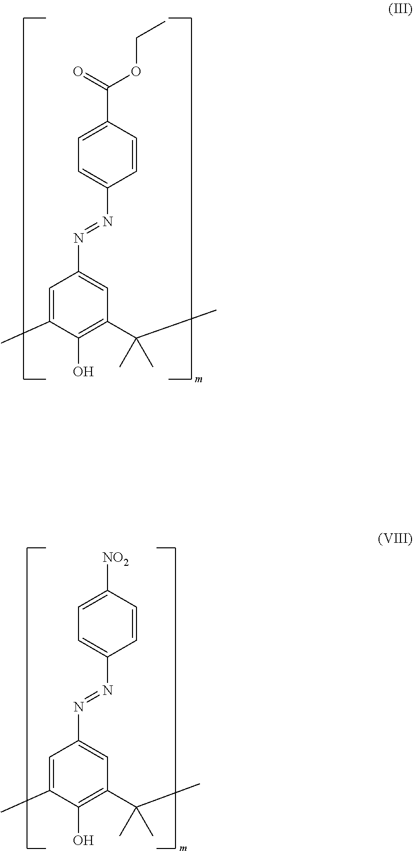

Preferably, m is 4.

In accordance with a second aspect, there is provided a method of producing a probe, the method comprising contacting an azo-calixarene with a salt.

The salt may be a fluoride salt or a carbonate salt.

Preferably, the method of the second aspect produces the probe of the first aspect.

The fluoride salt may comprise an ammonium fluoride salt, sodium fluoride or potassium fluoride, or a solvate thereof. The carbonate salt may be sodium carbonate or potassium carbonate.

Accordingly, the ammonium fluoride salt may be a salt of formula (IV):

(R¹³)₄NF (IV)

wherein, each R¹³ group is independently a C₁-C₁₂ alkyl. In one embodiment, each R¹³ group is independently a C₁-C₆ alkyl. Accordingly, each R¹³ group may independently be methyl, ethyl, propyl, butyl, pentyl or hexyl. In a preferred embodiment, each R¹³ group is n-butyl. Accordingly, the ammonium salt may be tetra-n-butylammonium fluoride.

Preferably, the method comprises dissolving the azo-calixarene and the salt in a solvent, and thereby contacting the azo-calixarene and the salt. Preferably, the method comprises dissolving the azo-calixarene and the fluoride salt in a solvent, and thereby contacting the azo-calixarene and the fluoride salt. The solvent may comprise dimethyl sulfoxide (DMSO), N,N-dimethylformamide (DMF), tetrahydrofuran (THF) or chloroform.

The concentration of the azo-calixarene may be as defined in relation to the first aspect. The concentration of the salt may be sufficient to obtain the concentration of the anion defined in relation to the first aspect.

Preferably, the azo-calixarene is as defined in respect to the first aspect. In one embodiment, the azo-calixarene is a compound of formula (II):

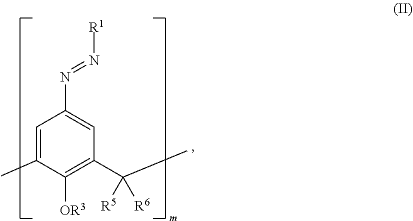

where R¹, R³, R⁵, R⁶ and m are as defined in relation to the first aspect. Preferably, m is 4, 5, 6, 7 or 8.

Prior to contacting the azo-calixarene with the fluoride salt, the method may comprise synthesising the azo-calixarene. Accordingly, the method may comprise contacting a compound of formula (V):

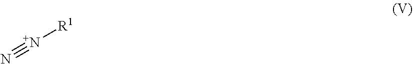

with a compound of formula (VI):

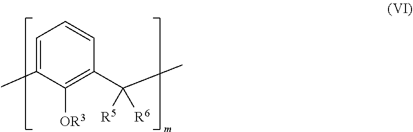

thereby synthesising the azo-calixarene.

Advantageously, the process does not require further purification. Accordingly, the probe may be produced quickly with a high yield.

The compounds of formula (V) and (VI) may be contacted at a temperature of between −20° C. and 30° C., more preferably at a temperature of between −15° C. and 20° C. or between −10° C. and 10° C., and most preferably between 0° C. and 5° C.

Preferably, the method comprises dissolving the compounds of formula (V) and (VI) in a solvent, and thereby contacting the compounds of formula (V) and (VI). The solvent may comprise a polar solvent. Preferably, the polar solvent comprises N,N-dimethylformamide (DMF) and/or an alcohol. Preferably, the alcohol comprises methanol.

Prior, or simultaneously, to contacting the compounds of formula (V) and (VI), the method may comprise synthesising the compound of formula (V). Accordingly, the method may comprise contacting a compound of formula (VII):

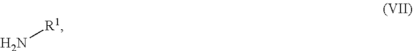

with a nitrite and thereby synthesising the compound of formula (V).

The nitrite may be sodium nitrite.

Preferably, the method comprises dissolving the compound of formula (VII) and the nitrite in a solvent, and thereby contacting the compound of formula (VII) and the nitrite. The solvent may comprise a polar solvent. Preferably, the polar solvent comprises water.

The solvent preferably comprises an acid. The acid may be hydrochloric acid.

In accordance with a third aspect, there is provided use of the probe of the first aspect to sense carbon dioxide.

The probe may be used to sense the quality of air in a building, a marine vessel or a greenhouse.

Alternatively, the probe may be used in a breathing apparatus. For instance, the probe could be used in a breathing apparatus configured to be used by a scuba diver. The probe can be used as an ‘active warning device’ designed to alert the diver when the CO₂ content of the breathing loop is approaching a dangerous level.

In accordance with a fourth aspect, there is provided use of the probe of the first aspect to capture carbon dioxide.

The probe may be used to capture carbon dioxide from the air in a building, a marine vessel or a greenhouse. Advantageously, the probe improves the air quality of the building, marine vessel or greenhouse.

Alternatively, the probe may be used to capture carbon dioxide from a landfill, power plant and/or mine. Advantageously, the probe prevents the carbon dioxide from being released to the atmosphere.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

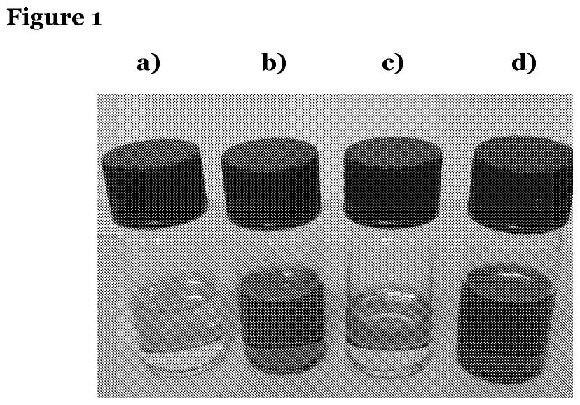

FIG. 1 is a picture showing the a) para-ester diazophenylcalix[4]arene (CA-AZ) in dimethyl sulfoxide (DMSO); b) para-ester diazophenylcalix[4]arene-fluoride (CA-AZ-F) in DMSO; c) CA-AZ-F in DMSO after having been exposed to CO₂; and d) CA-AZ-F in DMSO after having been exposed to CO₂ and then flushed with nitrogen (N₂) at room temperature;

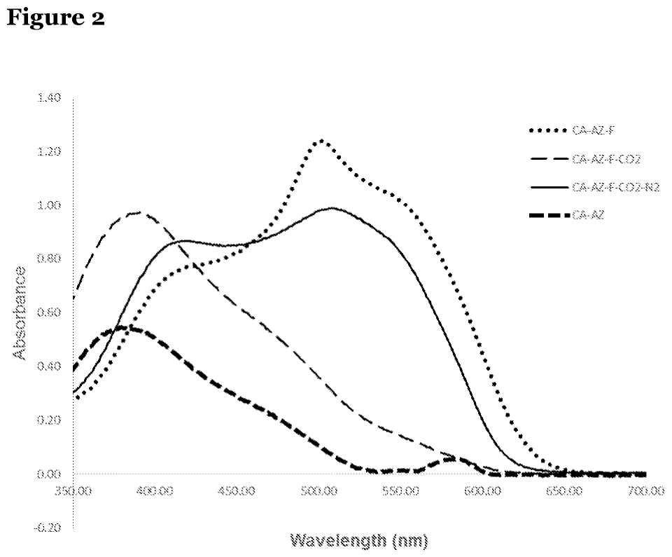

FIG. 2 shows the UV-vis spectra for each of the samples shown in FIG. 1;

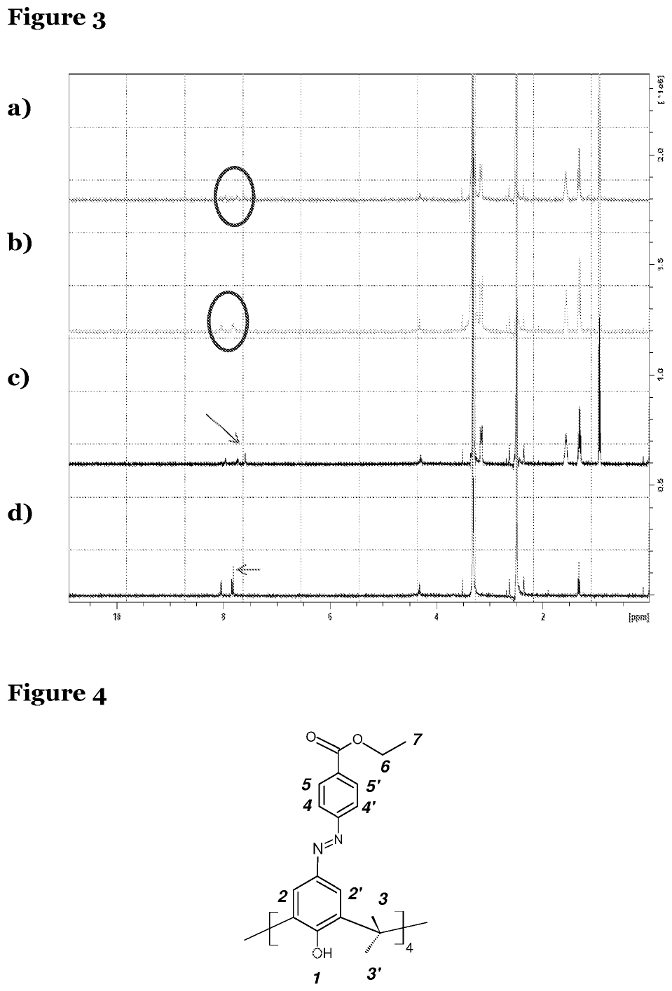

FIG. 3 shows the NMR spectra at 298.15 K for a) CA-AZ in deuterated DMSO (DMSO-d₆); b) CA-AZ-F in DMSO-d₆; c) CA-AZ-F in DMSO-d₆ after having been exposed to CO₂; and d) CA-AZ-F in DMSO-d₆ after having been exposed to CO₂ and then flushed with N₂ at room temperature;

FIG. 4 shows the structure of CA-AZ;

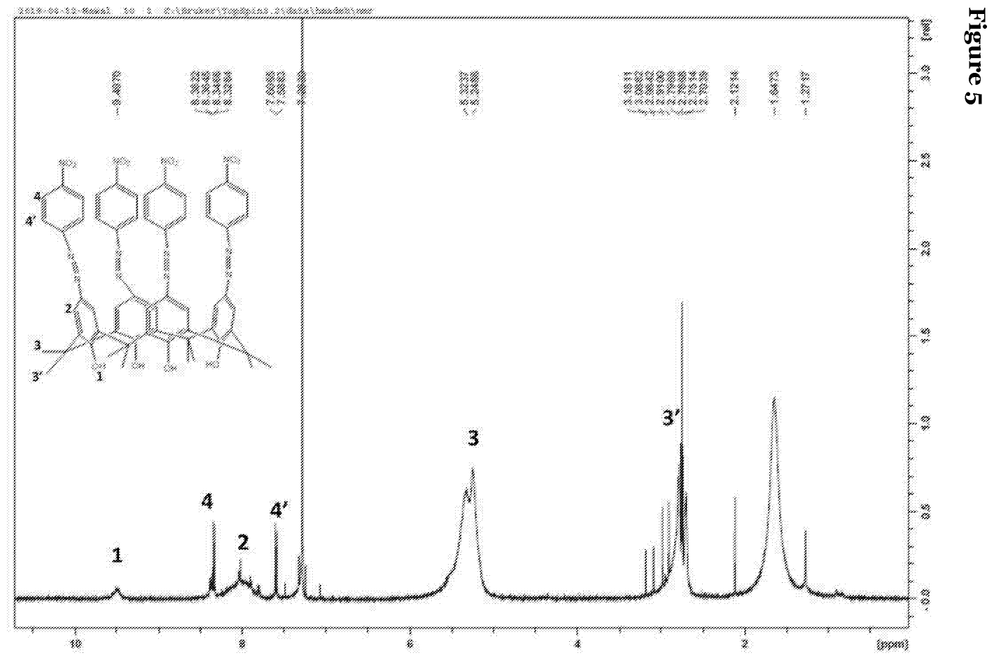

FIG. 5 shows the NMR spectra at 298.15 K for 5,11,17,23-tetrakis(4-nitrophenyeazocalix[4] arene (CA-AZ′) in deuterated Chloroform (CDCl₃);

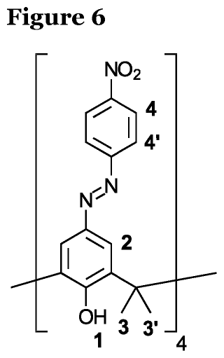

FIG. 6 shows the structure of CA-AZ′;

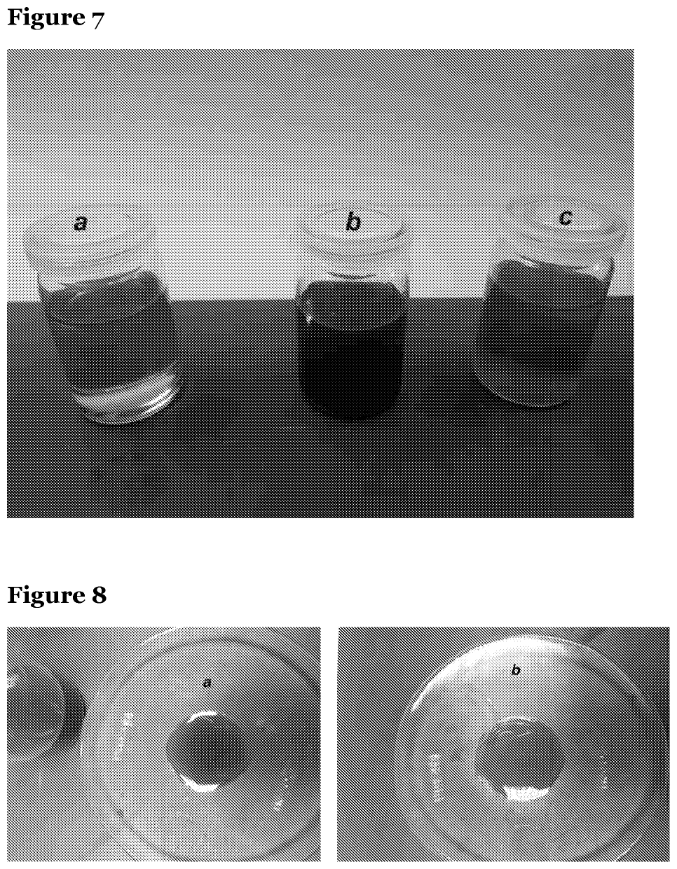

FIG. 7 is a picture showing the a) CA-AZ′ in DMSO; b) 5,11,17,23-tetrakis(4-nitrophenyl)azocalix[4] arene-carbonate (CA-AZ′-CO₃²⁻) in DMSO; and c) 5,11,17,23-tetrakis(4-nitrophenyl)azocalix[4] arene-fluoride (CA-AZ′-F) in DMSO; and

FIG. 8 is a picture showing a) CA-AZ′-CO₃²⁻ in DMSO; and b) CA-AZ′-CO₃²⁻ in DMSO after having been exposed to CO₂.

### EXAMPLE 1: SYNTHESIS OF CA-AZ

Materials

All chemicals used throughout the work were of analytical grade and were obtained from recognized chemical suppliers.

Salts used throughout the study were kept in vacuum oven and then stored in vacuum desiccators over phosphorus pentoxide, P₄O₁₀ for several days to remove water, before being used for experimental purposes.

Solvents

Methanol (CH₃OH), Sigma-Aldrich, HPLC grade, 99.7%.

N,N-Dimethylformamide (C₃H₇NO), Sigma-Aldrich, anhydrous 99.8%.

Deuterated Solvents Used in NMR Experiments

Chloroform-d (CDCl₃), Cambridge Isotope Laboratories, Inc, (D, 99.8%)+0.05% v/v TMS.

Dimethyl sulfoxide-d₆ (C₂D₆OS) Cambridge Isotope Laboratories, Inc. (D, 99.9%).

Analytical Reagents

Hydrochloric acid (HCl), Fisher Scientific, 35-38%.

Reagents Used in Synthesis without Further Purification

Ethyl 4-aminobenzoate (H₂NC₆H₄CO₂C₂H₅), Sigma-Aldrich, 98%, 112909.

Calix[4]arene-25,26,27,28-tetrol, Sigma-Aldrich, 95%.

Salts

Sodium ethanoate trihydrate (C₂H₃NaO₂.3H₂O), Sigma-Aldrich, ≥99%

Sodium nitrite (NaNO₂), Sigma-Aldrich, 97+ %.

Methods

In a 500 cm³ round-bottomed flask, ethyl 4-aminobenzoate (1.29 g, 7.8 mmol), sodium nitrite (0.41 g, 6.00 mmol) and conc. HCl (14 cm³) in water (25 cm³) was added gradually to a cold solution (0-5° C.) of 25, 26, 27, 28-tertrahydroxy calix[4]arene (0.64 g, 1.5 mmol) and sodium ethanoate trihydrate (1.17 g, 8.6 mmol) in a DMF/MeOH (2:1) mixture to obtain a dark orange coloured suspension. The mixture was stirred for 18 hours and a red precipitate was observed where the stirring was stopped. The mixture was filtered and the residue was washed with cold water then methanol several times. The product was left on a Schlenk line for one week. (98% yield).

The product was characterised by ¹H NMR (500 MHz) at 298 K.

¹H NMR (500 MHz, CDCl₃, δ in ppm); 10.25 (s, OH, 4H (1)); 8.13 &8.14 (d, Ar—H, 8H (5 & 5′)); 7.86 (d, Ar—H, 4h (4&4′)); 7.85 (s, Ar—H, 4H (2 & 2′)); 4.39 (q, COO—CH₂-CH₃, 8 H (6)); 4.4 (d, H-axial, 4H (3)); 3.87 (d, H-equatorial, 4H (3′)); 1.4 (t, CH₃, 12 H (7)).

Elemental analysis was carried out in duplicate at the University of Surrey; (C₆₄H₅₆N₈O₁₂) MW. (1129.20); Calculated %; C, 68.08; H, 5.0; N, 9.92. Found %; C, 68.14; H, 4.93; N, 10.2.

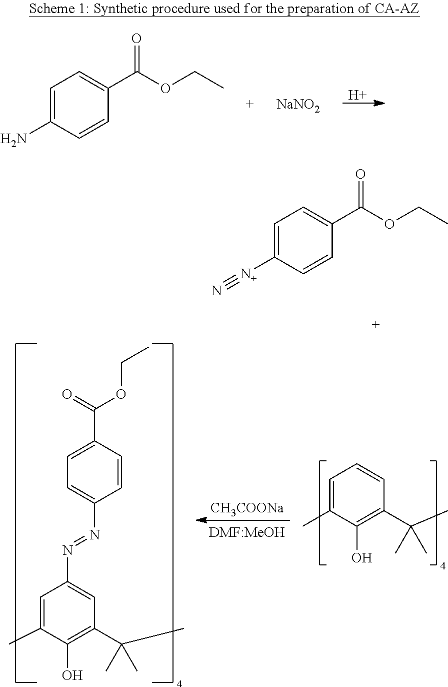

### EXAMPLE 2: DETECTION OF CO₂ USING PARA-ESTER DIAZOPHENYLCALIX[4]ARENE-FLUORIDE (CA-AZ-F) IN DMSO

Materials

Solvents

Dimethyl sulfoxide (C₂H₆OS), Fisher Scientific, 99%.

Salts

Tetra-n-butylammonium fluoride hydrate ((C₄H₉)₄NF.H₂O), Sigma-Aldrich, 98%.

Methods

CA-AZ prepared according to the method described in Example 1 was dissolved in dimethyl sulfoxide (DMSO) at a concentration of 10⁻⁵ mol·dm⁻³. Tetra-n-butylammonium fluoride (TBAF) was then added at a concentration of 10⁻³ mol·dm⁻³. Dry ice (solid CO₂) was then added to the solution. Finally, nitrogen gas (N₂) was bubbled through the solution for three minutes.

Results

Colour Change

As can be seen in FIG. 1, the addition of the TBAF to the CA-AZ solution induces a colour change from pale yellow to orange. When the solution comprising the CA-AZ-F complex was then exposed to CO₂ an immediate colour change from orange to yellow was observed. Finally, when the solution was purged with N₂ for three minutes the colour returned to orange.

This behaviour of CA-AZ was further investigated using a UV-vis spectrometer, and the results can be seen in FIG. 2. In particular, a bathochromic shift from 383 nm to 500 nm with a shoulder at 544 nm was obtained with F− addition to CA-AZ. When the solution was exposed to CO₂, the original spectrum of CA-AZ was substantially revived (λₘₐₓ=393 nm). However, the N₂ purge led to another bathochromic shift to 418 nm with a shoulder to 511 nm, where the spectrum pattern resembles the one of the solution comprising the CA-AZ-F complex. This suggests that the CA-AZ-F complex has reformed and CO₂ has been predominantly removed from the solution.

The inventors found that after purging with nitrogen, the solution can be reused to detect CO₂, and the colour changes and bathochromic shifts described above are again observed.

¹H NMR Studies

¹H NMR spectra were obtained for CA-AZ in DMSO-d₆, CA-AZ-F in DMSO-d₆, CA-AZ-F in DMSO-d₆ after having been exposed to CO₂ and CA-AZ-F in DMSO-d₆ after having been exposed to CO₂ and then flushed with N₂ at room temperature, and the results are shown in FIG. 3. The ¹H NMR chemical shift relative to the free ligand (the CA-AZ sample) was calculated and the results are provided in Table 1.

TABLE 1
¹HNMR chemical shift relative to the free ligand in
deuterated DMSO at 298 K

| | δ (ppm) | | | | | | | |
|---|---|---|---|---|---|---|---|---|
| | | H-2 | | | H-4 | H-5 | | |
| | | and | | | and | and | | |
| | H-1 | H-2′ | H-3 | H-3′ | H-4′ | H-5′ | H-6 | H-7 |
| δ _{Ref} | — | 7.82 | 3.51 | 4.3 | 7.87 | 8.05 | 4.3 | 1.32 |
| F⁻ | — | −0.23 | — | — | −0.18 | −0.26 | 0 | −0.39 |
| CO₂ | — | −0.02 | — | 0.1 | −0.01 | −0.01 | 0 | −0.39 |
| CO₂ | — | −0.23 | — | — | −0.18 | −0.26 | 0 | −0.39 |
| purged | | | | | | | | |
| with | | | | | | | | |
| N₂ | | | | | | | | |

FIG. 4 identifies the positions of H-1 to H-7. In all samples, the concentration of CA-AZ was 1×10⁻⁵ mol dm⁻³, and in the final three samples the concentration of the fluoride was 1×10⁻⁴ mol dm⁻³.

The peaks for H-3 and H-3′ protons did not appear in all of the spectra because they were too broad. Accordingly, it was not always possible to determine the shift for these aromatic protons.

Up-field shift in the aromatic protons (H-2, H-4, H-4′, H-5 and H-5′) was observed in the CA-AZ-F complex, and the relevant peaks are circled in FIGS. 3 (a) and (b). This suggests the deprotonation of the azophenol units in CA-AZ where the hydrogen is being abstracted from the —OH to the azo unit —N═N— resulting in the formation of —N . . . H . . . F—.

It will be noted that after exposure to CO₂ the ¹H NMR chemical shift for the aromatic protons was almost identical to that of CA-AZ in DMSO-d₆. This indicates that the fluoride ion is no longer complexed with the CA-AZ ligand. It is noted that while the chemical shifts for CA-AZ aromatic protons were substantially returned to their original positions after addition of CO₂, the peaks were broader than they were for the CA-AZ ligand, suggesting the formation of nucleophilic [CA-AZ]⁻.

Up-field shift in the aromatic protons was noticed again after the solution had been purged with N₂. This indicates that the CA-AZ ligand was once again complexed with the fluoride ion.

In conclusion, the ¹H NMR findings are in agreement with the UV-vis results.

Thermodynamic Studies of CA-AZ-F Complex Interacting with CO₂ in DMSO at 298.15 K

Thermodynamic parameters of complexation of CA-AZ with the fluoride and complexation of CA-AZ-F with CO₂ in DMSO at 298.15 K are listed in Table 2.

TABLE 2
Thermodynamic parameters of complexation
of CA-AZ with the fluoride
and complexation of CA-AZ-F with CO₂ in DMSO at 298.15K

| | | | | | Δ_{c}S° |
|---|---|---|---|---|---|
| | | | Δ_{c}G° | Δ_{c}H° | (J mol⁻¹ |
| Guest | ligand:guest | log K_{S} | (kJ mol⁻¹) | (kJ mol⁻¹) | K⁻¹) |
| F⁻ | 1:1 | 5.9 ± 0.1 | −33.7 ± 0.2 | −12 ± 0.3 | 72 |
| CO₂ | 1:1 | 5.76 ± 0.07 | −32.9 ± 0.3 | −38.4 ± 0.3 | −18 |

The stability constant (log Kₛ) of CA-AZ-F complex is quite similar to the stability constant value of the CA-AZ-F complex with CO₂, However, the complexation process of the anion probe with CO₂ is enthalpically controlled whereas the case of F⁻ complexation, both enthalpy and entropy contribute favourably to complex stability.

### EXAMPLE 3: DETECTION OF CO, USING A SOLID PROBE

Materials

Solvents

Tetrahydrofuran (C₄H₈O), Fisher Scientific, ≥99.85%.

Salts

Tetra-n-butylammonium fluoride hydrate ((C₄H₉)₄NF.H₂O), Sigma-Aldrich, 98%.

Methods

CA-AZ prepared according to the method described in Example 1 was dissolved in tetrahydrofuran (THF) at a concentration of 10⁻⁵ mol·dm⁻³. Tetra-n-butylammonium fluoride (TBAF) was then added at a concentration of 10⁻³ mol·dm⁻³. The resultant solution was poured into a dust free Pyrex Petri dish and the solvent was evaporated off at room temperature yielding a dark red film.

The dried probe was exposed to carbon dioxide at different time intervals.

Results

As the dried probe was exposed to the carbon dioxide a continual change in the colour of the probe from maroon red to red to orange and then yellow was observed as a result of the gas exposure

### EXAMPLE 4: SYNTHESIS OF 5,11,17,23-TETRAKIS(4-NITROPHENYL)AZOCALIX[4] ARENE (CA-AZ′)

The synthetic procedure is similar to the one given for example 1.

In a 500 cm³ round-bottomed flask, 4-nitroaniline (5.52 g, 40 mmol), sodium nitrite (1.69 g, 25 mmol) and conc. HCl (7 cm³) in water (25 cm³) was added gradually to a cold solution (0-5° C.) of 25, 26, 27, 28-tertrahydroxy calix[4]arene (4.24 g, 10 mmol) and sodium ethanoate trihydrate (4.08 g, 30 mmol) in a DMF/MeOH (8:5) mixture to obtain a red coloured suspension. The mixture was stirred for 18 hours; a red precipitate was observed where the stirring was stopped. The mixture was filtered and the residue was washed with cold water then methanol several times. Crystallisation was performed using DMF-Methanol.

Results

The structure of CA-AZ′ is shown in FIG. 6 and the 1H NMR spectrum is shown in FIG. 5.

¹H NMR (500 MHz, CDCl₃, δ in ppm); 9.49 (s, OH, 4H (1)); 7.58 (d, Ar—H, 8H (4′)); 8.1 (s, Ar—H, 4H (2)); 8.34 (d, Ar—H, 8H (4)); 5.24 (d, H-axial, 4H (3)); 2.91 (d, H-equatorial, 4H (3′)).

### EXAMPLE 5: PREPARATION OF 5,11,17,23-TETRAKIS(4-NITROPHENYL)AZOCALIX[4] ARENE-CARBONATE (CA-AZ′-CO₃²⁻) IN DMSO AND 5,11,17,23-TETRAKIS(4-NITROPHENYL)AZOCALIX[4] ARENE-FLUORIDE (CA-AZ′-F) IN DMSO

Materials

Solvents

Dimethyl sulfoxide (C₂H₆OS), Fisher Scientific, 99%.

Salts

Potassium carbonate and sodium fluoride.

Methods

CA-AZ′ prepared according to the method described in Example 4 was dissolved in dimethyl sulfoxide (DMSO) at a concentration of 10⁻⁵ mol·dm⁻³. Potassium carbonate or sodium flouride was then added at a concentration of 10⁻³ mol·dm⁻³.

Results

As can be seen in FIG. 7, the addition of the potassium carbonate to the CA-AZ′ solution induces a colour change from pale yellow to dark brown. Similarly, the addition of the sodium fluoride to the CA-AZ′ solution induces a colour change from pale yellow to dark orange.

### EXAMPLE 6: DETECTION OF CO₂ USING CA-AZ-CO₃²⁻ IN DMSO

A solution of CA-AZ-CO₃²⁻ in DMSO was prepared according to example 5. Dry ice (solid CO₂) was then added to the solution.

Results

Colour Change

As can be seen in FIG. 8, when the solution comprising the CA-AZ′-CO₃²⁻ complex was exposed to CO₂ an immediate colour change from dark brown to yellow was observed.

Conclusion

The inventors have shown that it is possible to use a probe comprising an azo-calixarene complexed with an anion. In particular, the inventors have synthesised para-ester diazophenylcalix[4]arene-fluoride (CA-AZ-F), 5,11,17,23-tetrakis(4-nitrophenyl)azocalix[4] arene-carbonate (CA-AZ′-CO₃²⁻) and 5,11,17,23-tetrakis(4-nitrophenyl)azocalix[4] arene-fluoride (CA-AZ′-F), and shown that these complexes can be used to detect and sense CO₂. The inventors have shown that the probe may be used in solution or as a solid.

Since the probe changes colour upon exposure to CO₂, the presence of CO₂ can be confirmed by the naked eye. Furthermore, the inventors have shown that the captured CO₂ can be recovered from the probe without the need to use elevated temperatures. This enables the probe to be used multiple times.

1. A probe comprising an azo-calixarene complexed with an anion.

2. The probe according to claim 1, wherein the anion is a fluoride ion or a carbonate ion.

3. The probe according to claim 1, wherein the probe comprises at least 1 mole of the anion for each mole of azo-calixarene.

4. The probe according to claim 1, wherein the azo-calixarene complexed with the anion is dissolved in a solvent.

5. The probe according to claim 4, wherein the amount of the azo-calixarene dissolved in the solvent comprises a concentration of between 1×10⁻⁶ and 1×10⁻³ mol dm⁻³ of the azo-calixarene.

6. The probe according to claim 4, wherein the amount of the anion dissolved in the solvent comprises a concentration of between 1×10⁻³ and 0.1 mol dm⁻³ of the anion.

7. The probe according to claim 1, wherein the probe comprises a solid.

8. The probe according to claim 1, wherein the azo-calixarene is an azo-calix[4]arene.

9. The probe according to claim 1, wherein the azo-calixarene is a compound of formula (I): 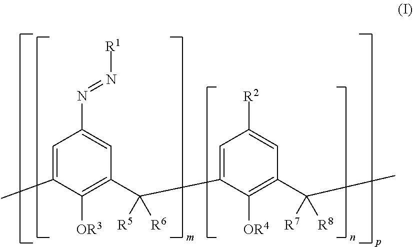
wherein R¹ is selected from the group consisting of hydrogen, an optionally substituted C₁-C₁₀ alkyl, an optionally substituted C₂-C₁₀ alkenyl, an optionally substituted C₂-C₁₀ alkynyl, NR¹¹R¹², an optionally substituted C₅-C₁₀ aryl and an optionally substituted 3 to 10 membered heteroaryl;
R² is selected from the group consisting of hydrogen, an optionally substituted C₁-C₁₀ alkyl, an optionally substituted C₂-C₁₀ alkenyl, an optionally substituted C₂-C₁₀ alkynyl, a halogen, OH, (CR⁹R¹⁰)ₐSO₂OR⁹, (CR⁹R¹⁰)ₐSO₂NR⁹R¹⁰, NO₂, (CR⁹R¹⁰)ₐPO₂OH, (CR⁹R¹⁰)ₐCOOR⁹, (CR⁹R¹⁰)ₐSS(CR⁹R¹⁰)_{b}COOR⁹, (CR⁹R¹⁰)ₐNR⁹R¹⁰ and N═NR⁹;
R³ and R⁴ are each independently selected from the group consisting of hydrogen, an optionally substituted C₁-C₁₀ alkyl, an optionally substituted C₂-C₁₀ alkenyl, an optionally substituted C₂-C₁₀ alkynyl, (CR⁹R¹⁰)ₐNR⁹R¹⁰), (CR⁹R¹⁰)ₐOR⁹, (CR⁹R¹⁰)ₐCOR⁹, (CR⁹R¹⁰)ₐCOOR⁹, (CR⁹R¹⁰)ₐCONR⁹R¹⁰, (CR⁹R¹⁰)ₐSO₂OR⁹, (CR⁹R¹⁰)ₐCOO(CR⁹R¹⁰)_{b}OR⁹, (CR⁹R¹⁰)ₐCOO(CR⁹R¹⁰)_{b}COR⁹ and (CR⁹R¹⁰)ₐCOO(CR⁹R¹⁰)_{b}SR⁹;
R⁵, R⁶, R⁷ and R⁸ are each independently selected from the group consisting of hydrogen, a halogen, an optionally substituted C₁-C₁₀ alkyl, an optionally substituted C₂-C₁₀ alkenyl and an optionally substituted C₂-C₁₀ alkynyl;
the or each R⁹ and R¹⁰ are independently selected from the group consisting of hydrogen, an optionally substituted C₁-C₁₀ alkyl, an optionally substituted C₂-C₁₀ alkenyl, an optionally substituted C₂-C₁₀ alkynyl, NR¹¹R¹², an optionally substituted C₅-C₁₀ aryl and an optionally substituted 3 to 10 membered heteroaryl;
an optionally substituted C₅-C₁₀ aryl or an optionally substituted 3 to 10 membered heteroaryl is unsubstituted or substituted with one or more substituents selected from the group consisting of an optionally substituted C₁-C₁₀ alkyl, an optionally substituted C₂-C₁₀ alkenyl, an optionally substituted C₂-C₁₀ alkynyl, a halogen, ORE, NO₂, CN, COOR¹¹ and NR¹¹R¹²;
each R¹¹ and R¹² are independently selected from the group consisting of hydrogen, an optionally substituted C₁-C₁₀ alkyl, an optionally substituted C₂-C₁₀ alkenyl and an optionally substituted C₂-C₁₀ alkynyl;
an optionally substituted C₁-C₁₀ alkyl, an optionally substituted C₂-C₁₀ alkenyl or an optionally substituted C₂-C₁₀ alkynyl is unsubstituted or substituted with one or more substituents selected from the group consisting of halogen, OH, NH₂, CONH₂, COOH, CN, a C₅-C₁₀ aryl, a 3 to 10 membered heteroaryl, a C₃-C₆ cycloalkyl and a 3 to 8 membered heterocycle;
a and b are each independently an integer between 0 and 6; and
m is an integer between 1 and 8; n is an integer between 0 and 7; and p is an integer between 1 and 4; wherein the total of (m+n)xp is an integer between 4 and 8;
or a salt, solvate or tautomeric form thereof.

10. The probe according to claim 9, wherein R¹ is an optionally substituted C₅-C₁₀ aryl or an optionally substituted 3 to 10 membered heteroaryl.

11. The probe according to claim 10, wherein R¹ is a C₅-C₁₀ aryl or a 3 to 10 membered heteroaryl wherein the aryl or heteroaryl are substituted with COOR¹¹ and/or NO₂.

12. The probe according to claim 9, wherein R³ is selected from the group consisting of hydrogen, an optionally substituted C₁-C₁₀ alkyl, an optionally substituted C₂-C₁₀ alkenyl and an optionally substituted C₂-C₁₀ alkynyl.

13. The probe according to claim 9, wherein R⁵ and R⁶ are each independently selected from the group consisting of hydrogen, an optionally substituted C₁-C₁₀ alkyl, an optionally substituted C₂-C₁₀ alkenyl and an optionally substituted C₂-C₁₀ alkynyl.

14. The probe according to claim 9, wherein the azo-calixarene is a compound of formula (II): 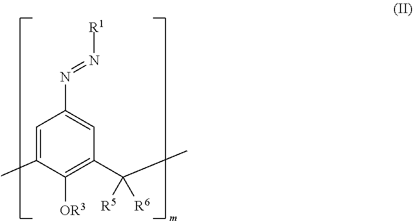
wherein m is an integer between 4 and 8.

15. The probe according to claim 14, wherein the azo-calixarene is a compound of formula (III) or (VIII):

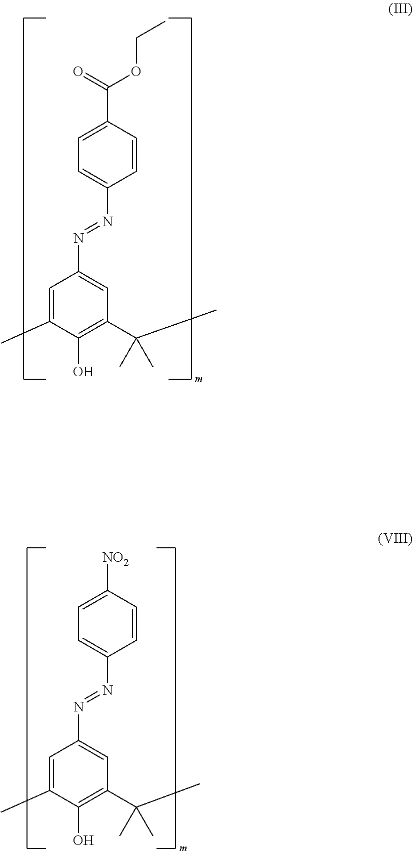

16. A method of producing a probe, the method comprising contacting an azo-calixarene with a salt.

17. The method according to claim 16, wherein prior to contacting the azo-calixarene with the salt, the method comprises contacting a compound of formula (V): 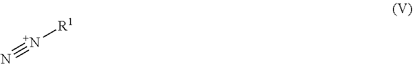
with a compound of formula (VI): 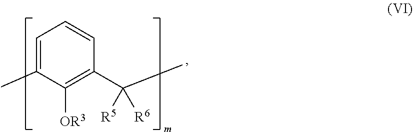
thereby synthesising the azo-calixarene,
wherein R¹, R³, R⁵, R⁶ and m are as defined in claim 9.

18. The method according to claim 17, wherein prior, or simultaneously, to contacting the compounds of formula (V) and (VI), the method comprises contacting a compound of formula (VII): 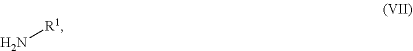
with a nitrite and thereby synthesising the compound of formula (V).

19. A method of sensing or capturing carbon dioxide, the method comprising using the probe as defined in claim 1 to sense or capture carbon dioxide.

20. (canceled)
